(19) United States
(12) Patent Application Publication
(10) Pub. No.: US 2005/0026148 A1
(43) Pub. Date: Feb. 3, 2005

(54) Method for the biochemical detection of analytes

(76) Inventors: Ulrich Rexhausen, Riedstr (DE); Manfred Wick, Wachterstr (DE)

Correspondence Address: NATH & ASSOCIATES, 1030 15th STREET, NW, 6TH FLOOR, WASHINGTON, DC, 20005, US

(21) Appl. No.: 10/478,412

(22) PCT Filed: May 23, 2002
(86) PCT No.: PCT/DE02/01875

(30) Foreign Application Priority Data
May 23, 2001 (DE) 101 27 221.9
May 23, 2001 (DE) 101 27 220.0

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

The invention relates to a method for detecting and/or quantifying analytes from a sample on an analysis carrier that has been formatted using a digital data code. Detection fields comprising the sensor elements required for the respective detection process, together with additional data structures in a defined digital format, are provided on the analysis carrier and combined to form sequcnces of formatted structures that can be interpreted as code words. To detect and quantify an analyte in a sample, the latter is applied to the analysis carrier and the formation of signal-generating elements is initiated at locations of molecular interaction. The localisation of signal-generating elements in the respective detection fields causes a formatted structure at this location to be replaced by another. This leads to the conversion of one code word into another within the predetermined quantity of valid code words. Both code words can be sequentially read and interpreted in the predetermined format. The statement concerning a successful or unsuccessful reaction is based on a comparison of the respective code words prior to and after detection. Detection takes place using a reading device, which is preferably constructed from components of the consumer goods industry.

### BRIEF DESCRIPTION OF THE INVENTION

The invention relates to a method for detecting and/or quantifying molecules from a sample on an analysis support formatted with a digital data code, wherein a detection reaction induces a change of the codewords, and these can be sequentially read and interpreted in the predetermined format.

Surface-based detection methods have been established for many years in the biochemical laboratory. With the increasing demands of molecular biological research, the need for highly parallelized and miniaturized technologies for studying the binding in complex molecular mixtures is growing.

The known methods for detecting and quantifying target molecules from a sample involve a plurality of steps, which are carried out by means of various devices which are sometimes elaborate and expensive. Microarrays are one of the possible ways of analyzing a multiplicity of biological molecules. Microarray technology, in which many different biological biomolecules such a DNA or proteins are applied, densely packed, in a predefined pattern on a substrate surface, has now become the standard method for parallel analysis of biological samples. This technology is used, for example, in the analysis of gene expression, in genetic diagnosis, in biological and pharmaceutical research and for the determination of genetically modified organisms in the food industry.

In microarray technology, biochemical sensor molecules such as DNA or proteins are applied to metal, glass, membrane or plastic surfaces, especially polycarbonate supports. After contact with the applied sample, it is possible to detect the molecular interaction and usually to obtain information about the bound quantity and/or about the strength of the interaction.

According to the prior art, the binding is usually detected via the generation and detection of an optical signal. In this case, it is conventional to use a microscope or a functionally similar device, especially a CD reader head (WO00/26677, WO00/36398). The information about binding which has or has not taken place at a particular site is obtained by image processing, which is typically carried out by computer software after analog-digital conversion in the case of microarrays with a relatively high density.

In one of the known methods, the information about binding which has or has not taken place at a particular site is labeled by the accumulation of grains (beads) at the site of the reaction of the analyte with the carrier-bound sensor molecule (for example EP918885 and Taton, Mirkin, Letsinger, Science 289: 1757-1760 (2000)). According to the prior art, beads bound in such a way are either identified directly as bodies or cause a chemical reaction such as a color change or a dyestuff precipitate. These are essentially detected by photometric methods. A camera and microscope combination is used to generate data, which can be evaluated by image analysis in the computer.

One of the disadvantages of the known methods for evaluating microarray analyses is the use of complicated and expensive devices and software for detecting and evaluating very weak signals, for example emission by a few molecules of fluorescent dyestuffs. These readers and evaluation devices are the result of many years of development work, use elaborate methods such as confocal scanning microscopy, and are very expensive. These devices furthermore require special software for identifying and localizing molecule spots and for interpreting and integrating detected signals.

In the next few years, initial results from gene research will start to be used in medical diagnosis and prognosis. Above all, simple and fast molecular detection methods will be required for various clinical problems. There is therefore a need for a simple, inexpensive and at least partly automated method for detecting and analyzing molecules in complex mixtures. It is an object of the present invention to provide a fast and simple method for analyzing and detecting analytes, which offers reliable interpretation of the results according to defined criteria as well as quantitative information.

In order to achieve this object, the invention relates to a method with the features mentioned in claim 1. Refinements of the invention are the subject matter of dependent claims which, like the abstract, have been worded with reference to the content of the description.

The above object is achieved according to the invention by a method in which a molecular interaction at a particular site on the support leads to the generation of detectable structures, referred here as signaling elements, which can be read and interpreted in the context of the format defined in the form of a digital data code on the analysis support.

Detection fields with the sensor elements needed for the respective detection, as well as other data structures, are applied in a digital format on the analysis support and are combined to form sequences of format structures which can be clearly interpreted as codewords. In order to detect or quantify an analyte in a sample, the latter is applied to the analysis support and modulation of the digital signal level on the respective detection fields is initiated with the aid of a signaling element. In this way, one codeword is replaced by another codeword allowed within the set of valid codewords. The information about a reaction which has or has not taken place is based on comparison of the respective format structures before and after the detection. The exemplary embodiments are a CD, a magnetic card, an optical card and a barcode card.

The method according to the invention has the advantage, over known methods, that elaborate two-dimensional image analysis since digital data are obtained. The provision of a digital information structure obviates analog signal processing, which is complicated and prone to errors. Reliable interpretation of the results according to defined criteria is facilitated. The analysis support and the reader, which is constructed from standard components in the consumer goods industry, can furthermore be adapted to one another easily as a function of the problem, so that various tests can be developed and produced quickly and inexpensively in mass production.

Other advantages, features and possible applications of the invention will be described below with the aid of the detailed description and exemplary embodiments, with reference to the drawings. In the drawings:

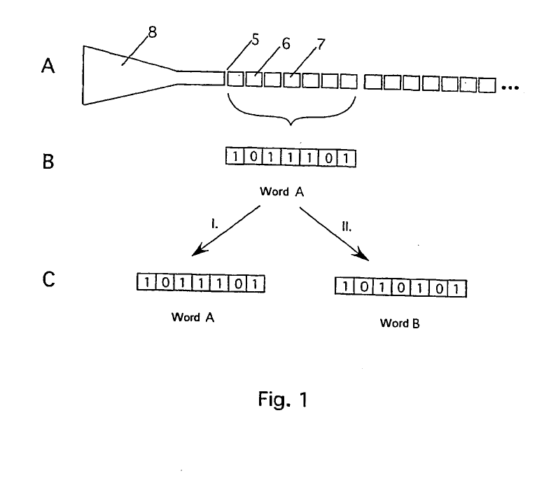

FIG. 1: A) shows a schematic representation of an exemplary sequence of format elements on an analysis support, which consists of blank fields 5, address fields 6 (gray squares) and detection fields 7 (white squares), and which forms a track. The header region 8 is used for finding and initializing the track when reading; B) before detection, the sequence of format elements represents the codeword A; C) after detection has taken place, the represented sequence of format elements may represent the word A, if no reaction takes place (I.), or the codeword B in the event of a reaction (II.). The binary codewords A and B differ by a change of the signal level at the fourth position, which corresponds to a detection field.

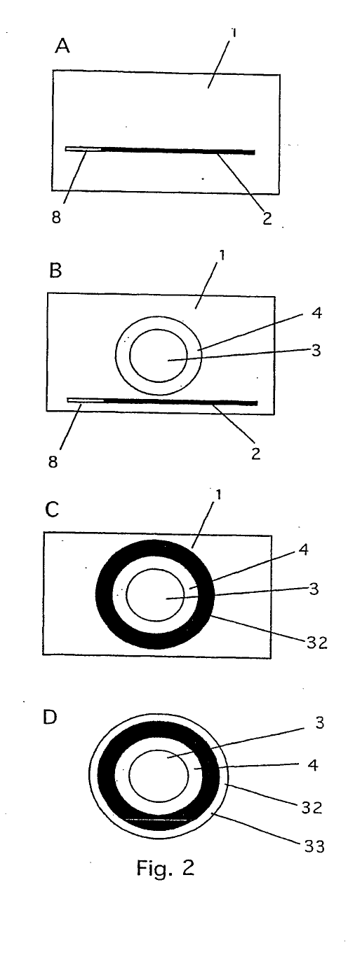

FIG. 2: A) shows a schematic representation of the simplest embodiment of the analysis support 1, with a track 2 of format elements, sketched linearly by way of example, and a header region 8; B) shows a schematic representation of another possible configuration of the analysis support 1 with a track 2 of format elements and a header region 8, an optional central hole 3 for combination of the analysis support and a CD, together with a spiral data track 4 for the data storage and software; C) shows a schematic representation of an example of another preferred embodiment of the analysis support 1 with a central hole 3, a spiral data track 4 in the CD-R standard for the data storage and software, and a spiral track 32 of format elements in the CD-R standard; D) shows a schematic representation of another possible configuration of the analysis support in the form of a CD 33 with a central hole 3, a spiral data track 4 in the CD-R standard for the data storage and software, and a spiral track 32 of format elements in the CD standard.

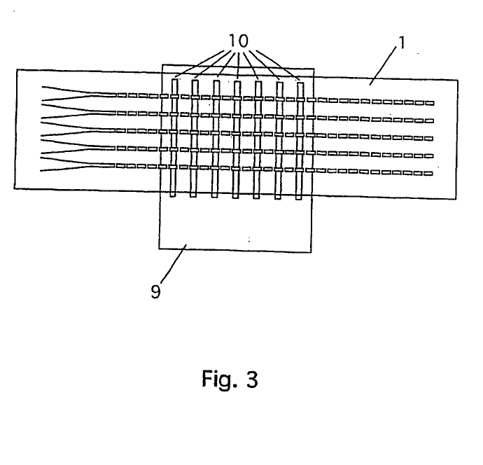

FIG. 3: shows a schematic representation of an analysis support 1 with a plurality of tracks arranged in parallel, which consist of rows of detection, information and blank fields. Exemplary filling of the analysis support 1 by means of a microfluidic plate 9 with sensor elements or analyte solutions through microchannels 10 embedded in the support, or otherwise spatially arranged 10, is represented.

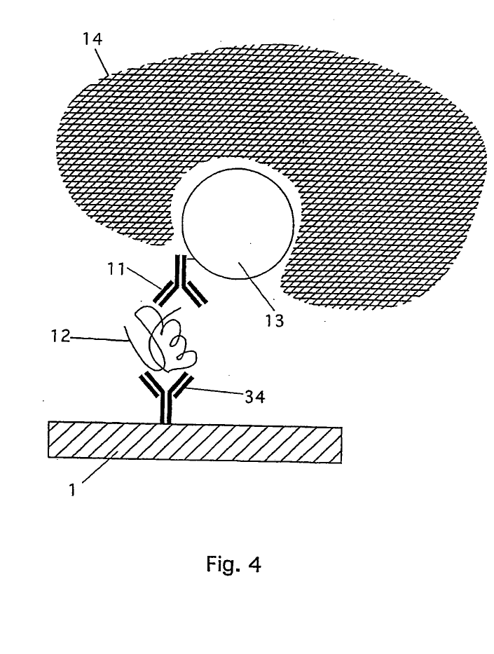

FIG. 4: shows a schematic representation of a detection according to Example 1. A binding reaction between a sensor element 34, here an antibody, applied to the analysis support 1 and an analyte molecule 12, here a protein, from the sample is represented. The detection is carried out in a sandwich immunoassay with the aid of a second antibody 11, which is directed against a different epitope of the protein. A colloidal gold particle 13 coupled to the second antibody leads to the deposition of a silver grain 14, which is used as a signaling element.

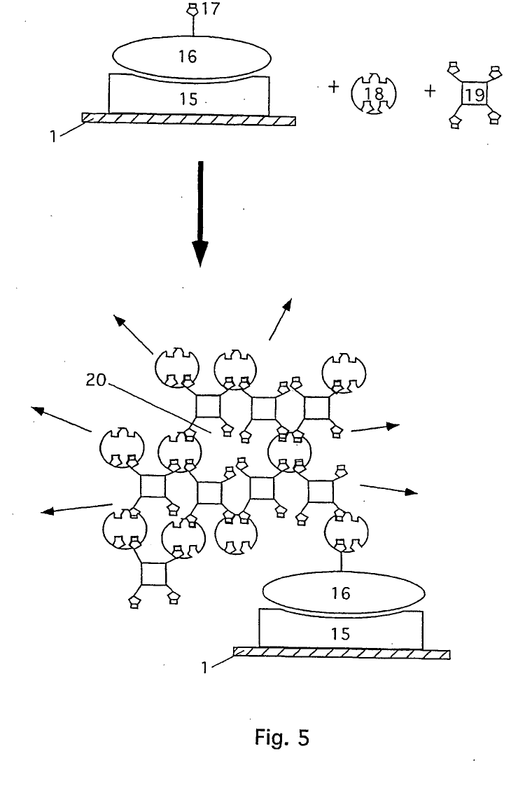

FIG. 5: shows a schematic representation of a detection according to Example 3. A binding reaction between a receptor 15 applied to the support 1 and a ligand 16 from the sample, which is coupled to a hapten 17, here biotin, is represented. After addition of a mixture of streptavidin 18 and biotinylated ferretin 19, signaling elements are produced in the form of molecular complexes 20.

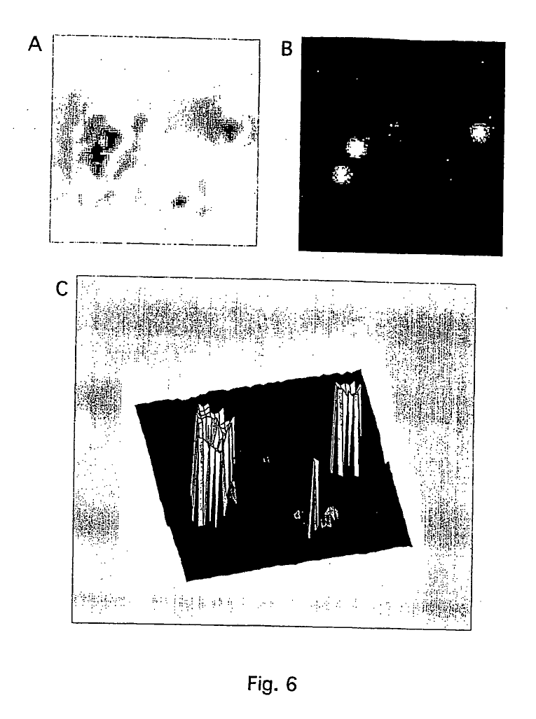

FIG. 6: shows signaling elements for the example of polystyrene beads with a diameter of 1 μm, which are detected according to two different methods and interpreted in binary form. A) shows an image recorded by a CD reader head; B) shows the same beads recorded by fluorescence microscopy; C) shows a three-dimensional representation of the data from A), which can be interpreted in binary form.

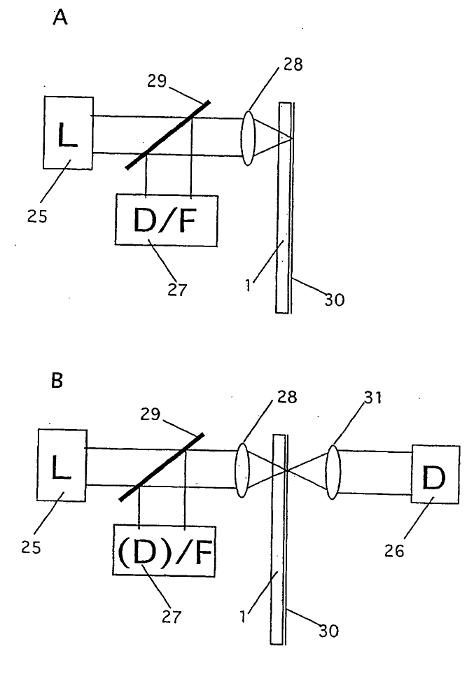

FIG. 7: A) shows a simplified schematic representation of an optical system for detecting the reflection signal, which represents a conventional CD reader head, consisting of a laser (L) 25, a detector for focal adjustment and signal detection (D/F) 27, a focusing instrument 28 and a semisilvered plate 29. The analysis support 1 with the silvering 30 is represented in the beam path in the side view; B) shows a simplified schematic representation of an optical system for detecting the transmission signal, consisting of a CD pickup from Example A), the actual detector for the transmission measurement (D) 26 and optionally an additional focusing instrument 31. The analysis support 1 with the silvering 30 is represented in the beam path between the focusing instruments 28 and 31 in the side view.

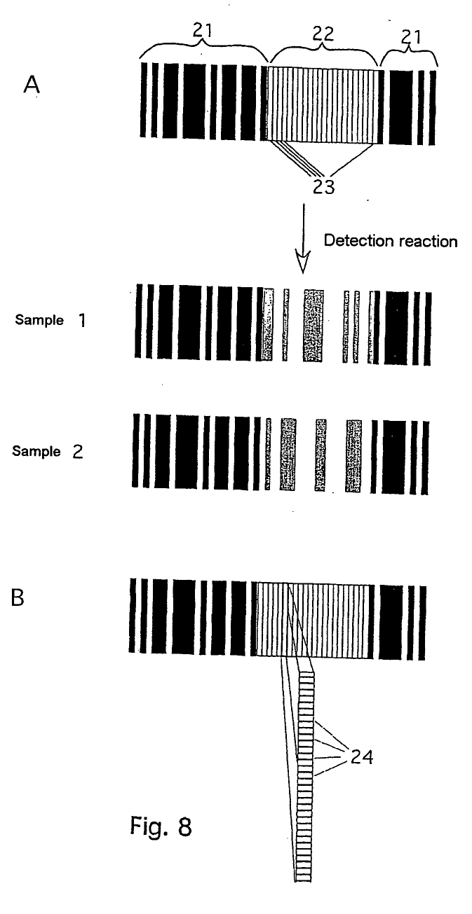

FIG. 8: shows a schematic representation of an analysis support, on which the detection fields are applied as parallel strips so that they can be read using a barcode reader. A) The individual detection fields 23 are arranged in relatively large detection regions the form of test strips 22. They may be fitted in a test unit together with address fields 21 which, for example, contain information about test specifications, codings (dongle) or product identification. After a detection reaction with analytes from different samples 1 and 2, different patterns of signaling elements are obtained on the test strips, and these can be read using a barcode reader; B) The test strips 22 may consist of a plurality of detection fields 24, which are arranged orthogonally to the main reading direction and, for example, may contain graded concentrations.

FIG. 8: shows an image of fine lines in the micrometer range, which are attributable to an antibody-antigen reaction with subsequent silver deposition. A) shows an analog image recorded by a CD reader head; B) shows analog image lines from A), read along the horizontally dashed line; C) shows digital processing of the image line from B), produced from the analog signal by using a threshold criterion, identified by the horizontal line in B).

### METHOD FOR BIOCHEMICAL DETECTION OF ANALYTES DETAILED DESCRIPTION OF THE INVENTION

The present invention comprises the following essential components, which in combination constitute the preferred use of the method according to the invention:
an analysis support, consisting of a base support and blank, information and detection fields applied thereon, which are formatted in a digital data code;
sensor elements, applied to a defined pattern of the detection fields;
a standard protocol for carrying out specific interactions between sensor elements and analytes from the sample, and for the formation of signaling elements on the detection fields;
a reader for the detection of signaling elements on the detection field in the context of the predetermined digital format, together with control means and evaluation software.

The components essential for carrying out the method according to the invention will be explained in detail below:

Coding

The analysis support is formatted with a defined digital data code. The format is dictated by the specific technical embodiment and the detection system which is used, or in general the reader. For example, the format is established by the respective characteristics of pits and lands in CD technology, “high” and “low” levels in digital electronics, dots and dashes in Morse code etc. In the case of the detection systems known from the CD technology, for example, a defined geometry, length and arrangement of pits and lands on an optical disk, according to the Philips Red Book standard, determine the respective format.

The coding describes the control mechanism according to which the information is processed. A code is the sum of all valid codewords, each codeword being defined by a unique sequence of the predetermined format structures. Coding generally contains additional rules such as redundant error-correction information, for example cross summation or interleaving, as specified for example by the Red Book standard in the CD industry.

The most common formats in the consumer goods industry use a binary code. This case will be described in detail below as a preferred embodiment. A binary code is given by a particular sequence of “0” and “1” levels (or “high” and “low” levels) with a defined length. In the case of the present invention, the binary code consists of sequences of format structures, which are defined by “blank-field” and “information-field” format elements (FIG. 1A) with particular signal levels and signal lengths. Information fields may be address fields or detection fields.

In a particularly preferred embodiment, the sequences of format elements on the analysis support form one or more tracks. In a preferred embodiment, a track is divided into four different subregions (FIG. 1A):

1. Header region. Here, the track is found and initialized (tracking).

2. Blank fields. The breaks are essential in order to determine the position of the detection field to be read, by counting or via addressing. The blank fields have an invariant level (“0” or “1”) and a variable length.

3. Address fields. These contain structure information and position information, with which it is possible to assign the signal obtained by the molecular interaction to a defined substance. The address fields have an invariant level (“1” or “0”) and can have different lengths. The address fields and the blank fields have inverse levels. If the blank fields have a “0” level, for example, then the level of the address fields is “1”, or vice versa.

4. Detection fields. The sensor elements are applied here. The detection fields are characterized by a variable level (“0” or “1”) and can have different lengths. A level change from “0” to “1” or from “1” to “0” on a detection field indicates that detection of the analyte has taken place (see below).

Any conceivable defined arrangements of format elements in one or two dimensions are possible, and are therefore covered by the invention.

It is a fundamental concept of the present invention that the signaling elements on the detection fields can be adapted to the predetermined format, and that the result of respective biochemical detection can be obtained from comparison between the respective sequences of the format structures before and after the analysis is carried out.

According to the invention, the vocabulary of the code consists of a limited number of codewords with a predetermined length, which are made up of the “blank-field” and “information-field” format elements with defined signal levels and signal lengths. Each word may contain one or more detection fields. Undefined words are not allowed and are identified as errors by the interpreter software. Advantageously, a certain number of codewords have no detection fields and are used, for example, for separating and/or addressing sizable code blocks. The number of tests applied to a support can be scaled in a simple way by combination or concatenating the sequences of format elements.

In order to determine the detection result, a defined sequence of format elements defines two allowed codewords A and B, which differ by a change of the level from “0” to “1” or from “1” to “0” on at least one detection field (FIG. 1B). Before detection, the levels on the detection fields are such that the sequence of format elements represents the codeword A, for example. A detection reaction causes replacement of “0” by “1”, or vice versa. The codeword A is therefore converted into the codeword B. If no reaction has taken place on the detection fields, the respective sequence of format elements still represents the codeword A (FIG. 1C). The result is interpreted by comparison between the respective sequences of format structures before and after the analysis.

Detection evaluation on an analysis support according to the invention will be explained using the example of Morse code. Suppose that Morse code is implemented on the analysis support by a predetermined sequence of “low” and “high” levels. Assume that the “low” level always has the length 1 and is represented by the symbol “0”. In this example, it is used only as a spacer mark. The “high” level selectively has the length 1 or 3, however, and is consequently represented by “1” or “111”. Assume that the codeword A is defined by the sequence “1 0 1 1 1 0 1” and the codeword B is defined by the sequence “1 0 1 0 1 0 1” (FIG. 1B). Both words have the same total length of 7, but the level at the fourth position, which is here intended to correspond to a detection field on the analysis support, is different. A binding reaction on the detection field at the fourth position converts the “1” level at this point to a “0” level. The allowed word A is therefore changed into the allowed word B (FIG. 1C). The detection result is derived by simple comparison between the output word A obtained before the detection and the word B after the detection. The word A will be read if no reaction takes place (“negative test”), and the word B will be read in the event that a reaction has taken place (“positive test”). A different codeword C, or an undefined word, would be picked up in the scope of the interpretation and identified as an error. This feature of the method can advantageously be used for quality control.

This indirect obtaining of sequential binary data, based on coding, has substantial advantages over the known methods. First, elaborate two-dimensional image analysis is avoided. Secondly, the selection of a digital code establishes a digital information structure, which obviates complicated and error-prone analog signal processing. Thirdly, the coding of binary signals allows error detection and correction. Fourthly, standard modules or complete devices from the consumer goods industry may be used for the detection and evaluation.

A format which is conventional in the consumer goods industry is preferably used on the analysis support according to the invention. This may, for example, be audio CD, optical disk, CD-R, CD-RW or MO (ECMA 154, ISO/IEC 10090), CD-ROM (ECMA 130, ISO/IEC 10149), DVD-R (ECMA 268, ISO/IEC 16449) or subsequent standards.

The invention is not restricted to formats which use a binary code. Other formats based on digital code systems are also conceivable, and are therefore covered by the invention.

Multidimensional codes, such as two-dimensional barcodes, are a direct extension and are therefore included in the subject matter of the present invention. It may be advantageous to use such coding for biochemical detections because it allows parallel data processing, error correction and obtaining of redundant information. Parallel data acquisition, for example by means of a camera or CCD chip, is advantage in this case because the absolute positions can thereby be determined and interrogated directly. This contrasts with serial reading, in which position determination needs to be carried out with the aid of marks, addresses or synchronization.

Analysis Support

The analysis support of may have a polygonal, round, oval or other two- or three-dimensional shape. In a particularly preferred embodiment of the invention, a substrate which is similar in geometry and handling to conventional magnetic cards is used as a support (FIG. 2A). Magnetic and chip cards have enjoyed widespread use owing to their practical size and ease of handling. The essential features are their robustness and the possibility of accommodating a limited amount of information on a small space. In one common variant, the magnetic cards contain a linearly arranged data strip at a constant distance from one of its outer edges. In combination with the simple reader, this leads to simple mechanical handling—the card can even be swiped by hand through a reader. Because magnetic cards can be produced inexpensively in large numbers and the readers are easy to manufacture, this embodiment of the invention makes it possible to construct readers which are orders of magnitude less expensive in production and simpler to use than known equipment for the detection of biological analytes.

Magnetic storage media, optical cards, a barcode card (FIG. 8) or combinations thereof may also be used as analysis supports. According to the invention, the external shape is not restricted to the usual card standards. A conventional CD (FIG. 2D) or derivatives thereof, for example CD-R, DVD, may likewise be used as analysis supports. Existing formatting of format structures on the analysis support may advantageously be utilized, which obviates involved and expensive reprogramming.

The detection area may be part of the support or accommodated on a separate auxiliary support. Software, databases, signatures and other information, besides the sensor elements, for example test specifications, protocols for carrying out the respective test, etc. may be fitted in any desired configuration on the same support. Any desired combinations of conventional data storages, such as magnetic strips, card chips, barcodes, CD (FIGS. 2B, 2C and 2D), CD-ROM, audio CD or CD-R may be integrated in the support.

If the sequences of format elements on the analysis support form one or more tracks, and the detection, information and blank fields are equivalently dimensioned, and each have an area of 5×5 μm², then a length of 35 μm for the test unit is obtained in the Morse code example described above. When a plurality of such test units are arranged successively, they form a track (FIG. 1A). Assuming a track length of 6 cm, an individual analysis number of up to 2000 is achieved in one track. It is therefore possible either to carry out up to 2000 different tests on one analysis support or, by using graded concentrations, to obtain extensive statistics about a small number of detections.

It is possible to have a plurality of tracks of detection, information and blank fields arranged in parallel per support (FIG. 3). This arrangement offers the advantage of increasing the individual test number and parallelizing the test procedure and readout of the results. Furthermore, for example, the filling of individual detection fields with sensor elements and/or a sample can be parallelized via a microfluidic plate.

Linear, curved, radial, spiral and circular, or other geometrical or even stochastic arrangements of detection, information and blank fields are also possible, of course, and are therefore covered by the invention.

Polymer plastics with various physicochemical properties adapted to the analytical task, as well as glass, semiconductors, metals, metal alloys, ceramics, hybrid materials or combinations of these substances may be used as the support material. A particularly advantageous embodiment in combination with optical reading methods employs supports made of glass, transparent plastics or polymer materials, and especially optical-quality polycarbonate as used in the CD and DVD industry.

In order to produce an analysis support, basic formatting is first applied to the base support. For each specific test, the sensor elements necessary for the respective detection are then applied in a predefined pattern on the format elements provided as detection fields.

Sensor Elements

Any substances which may be of use for biochemical or medical detection can be used as sensor elements. Examples include sugars, steroids, hormones, lipids, proteins, in particular monoclonal or polyclonal or recombinant antibodies, peptides, antigens of any type, haptens, DNA, RNA, as well as natural and artificial derivatives thereof, in particular aptamers and PNA, but also organic-chemical active agent libraries as used, for example, in pharmacological research and development. Cells, microorganisms, viruses or parts thereof, preparations and extracts from biological materials, metabolites and the like may equally well be used as sensor elements. Further chemical, biological, organic or inorganic elements with sensor characteristics may equally well be employed, and are therefore covered by the invention.

According to the invention, a multiplicity of different sensor elements may advantageously be applied to a substrate surface. The sensor elements of one type are in this case respectively applied to defined detection fields in a spatially limited way. The extent of the detection fields in one or two dimensions may be less than 10 μm, preferably less than 2 μm and particularly preferably less than 1 μm. Besides the sensor elements, it is also possible for other molecules or signaling elements, which are used for calibrating or standardizing the analyses, to be applied according to a predefined arrangement on neighboring detection fields.

Solutions by which sensor elements can be bound to the support surface without detrimentally affecting their functionality are known to the person skilled in the art from the prior art. The sensor elements may be covalently or noncovalently bound to the support surface or applied to the support surface. The sensor elements may be applied mechanically to the support surface, in particular by droplet application, for example with the aid of inkjet printing or spotting, or by means of lithographic methods according to the prior art. The wetting of the detection fields with the sensor elements may also be carried out by means of channels, preferably microchannels or microfluidic networks (FIG. 3). Simple immersion of the analysis support in a liquid bath containing the sensor elements is also possible, after selective activation of detection fields or passivation of blank fields on a surface-activated support. A spin-coding method may likewise be used, for example in order to extensive activation of the surface.

In another advantageous variant, material which is transparent at a particular wavelength is used for the analysis support. The light-guiding properties of the support may be used according to the invention in order to couple or synthesize particular molecules at predefined sites via position-selective guiding of light. Synthesis controlled by electric fields directly on the support is likewise conceivable.

The sensor elements may, for example, be covalently linked by binding amino, thio or phospho groups, which are already present or have been specially introduced, to a terminal-group-functionalized silanized support surface. Alternatively, biotinylated sensor elements may be specifically immobilized on the support surface by means of streptavidin coating.

Signaling Elements

It is a fundamental concept of the present invention that the physical parameters of the signaling elements, such as size, shape and signal level, can be adapted to the format structure applied in the form of blank fields and address fields. This contrasts with the usual procedure, in which a format structure and device are developed so as to match the given signal. The constraints such as positioning and dimensioning of the format elements and of the signaling elements are dictated by the coding.

Any resonant processes (absorption, fluorescence, phosphorescence, plasmon resonance, quenching etc.) and nonresonant processes (reflection, diffraction, scattering etc.) from spectroscopy may be used for the signaling. Alternatively, electromagnetic effects (piezo, resonance shift, capacitance change, Hall effect, magnetic effects, electrical charge displacement etc.) may be used for the signaling. Chemical processes such as silver deposition, precipitation of oxides, oxidation or reduction of reagents, electroplating and the like may also be employed for the detection. Other chemical, physical or biological signalers may equally well be used, and are therefore covered by the invention.

A variety of commercially available substances and bodies, which can constitute or form detectable structures, may be used as signaling elements. They may be advantageously be microspheres (beads) (FIG. 6) of any shape and size, such as metal, magneto, silica or fluorescence-labeled beads, fluorescent or radioactive labels as well as molecular complexes or aggregates, layers of precipitates or dyestuffs.

In a preferred alternative embodiment of the optical detection, silver grains are formed on an initiator, in particular an electron donor such as metal-molecule or metal grains, coupled to analyte molecules, at the site of the interaction, which is usually binding (FIG. 4). In another preferred variant of the optical detection, molecular complexes are formed in a reaction between two or more different binding partners, for example one avidin or streptavidin, and another multiply biotinylated substance (FIG. 5). Biological objects of suitable size such as cells, bacteria, pollens, virus particles or parts thereof may advantageously be used as signaling elements.

In another advantageous embodiment, the resulting detectable structure may also be produced by initiation of a chemical reaction with another substance. To this end, another substance is applied to defined points on the support, in addition to the sensor element which is intended to interact with the sample to be analyzed. This substance is converted into a detectable structure at the site of the reaction by the interaction between an analyte and the associated sensor element. For example, an enzyme such as horseradish peroxidase (HRP) coupled to the analyte may initiate an enzymatic conversion of the substrate localized on the support into a signaling element through the binding of the analyte to the corresponding sensor element. The reaction is facilitated either by spatial proximity between the enzyme and the substrate, or by release of the substrate into the solution, during the interaction between the sensor element and the analyte or directly thereafter.

The properties after an interaction between a sensor element and an analyte give rise to detectable structures in the form of signaling elements, and the signal levels resulting therefrom correspond to the predetermined digital format on the support. These structures may be dimensioned by various methods known to the person skilled in the art. For example, saturation in the formation of signaling elements may be achieved through the stoichiometric ratios of the substance concentrations which are used, so that no further effective growth of the signaling element takes place after an intended size is reached. Alternatively, the reactions leading to the formation of detectable structures may be blocked in a time-controlled way. In particular, blocking substances such as inhibitors for enzymatic reactions, competitors such as biotin in the example of FIG. 5, or substances which break down free reactants, for example specific proteases, may be used for this. In a particularly preferred embodiment, the detection reactions take place in a continuous flow system, so that the reactants involved can be flushed away from the reaction sites with a buffer after an experimentally determined optimum reaction time, which is necessary in order to form detectable structures with intended dimensions. If catalytic reactions are involved, they may also be stopped by removing or blocking the catalyst. In the case of photodependent reactions, switching off the light source also leads to controlled termination of the reaction.

Detection fields and signaling elements with dimensions smaller than 10 μm, preferably smaller than 2 μm, and particularly preferably smaller than 1 μm, are advantageously formed. Any lower limit on the miniaturization is due only to the resolution of the detection unit.

Establishment of the reaction conditions and selection of the reactants leads to detectable structures in an interaction between a sensor element and an analyte, which can be read and digitally interpreted in the data format previously applied to the support. In this way, the result can be interpreted directly as “positive test” or “negative test” by comparing the signals before and after the detection with one another in the context of the predetermined formatting.

A molecular interaction may lead either to the generation of an additional signal or to reduction of the signal level due to the signaling element. The appropriate contrast methods, i.e. change of the signal level due to the signaling element, should in this case be selected as a function of the standard protocol for the respective biochemical test, the measurement method and the analysis support.

Successful detection of the analyte is characterized by a modified signal structure, i.e. a signal level change from “low” to “high”, or from “high” to “low”, and unsuccessful detection of the analyte is characterized by an unmodified signal. With the aid of the following examples, the four possible contrast methods for the preferred variant of the optical detection by means of reflection and transmission measurements will be described by way of example.

1. “Low”-“high” transition in a reflection measurement. This situation is encountered, for example, in the case of a weakly reflecting analysis support surface and a reflective signaling element. Such contrast may, for example, be produced by means of a black surface (“low” level) and by silver precipitation induced by the positive test as a signaling element (“high” level). A similar principle is used in black-and-white photography.

2. “High”-“low” transition in a reflection measurement. Here, for example, binding or production of a scattering body (“low”) as a signaling element on a silvered support surface (“high”) may be initiated by a successful reaction. Such a scattering body may, for example, be a bead or a silver grain.

3. “Low”-“high” transition in a transmission measurement. This situation is encountered, for example, in the case of a silvered analysis support and a signaling element in the form of a window. The window (“high”) may be produced by local etching, induced by a molecular interaction with the analyte, and associated removal of the mirror layer (“low”) from the detection fields. This method is comparable with the typical lithographic etching methods in semiconductor physics.

4. “High”-“low” transition in a transmission measurement. This situation is encountered, for example, in the case of an unsilvered and transparent analysis support and a reflective or scattering signaling element. Such a contrast may be produced, for example, with the aid of an uncoated glass plate (“high”) by means of silver precipitation as a signaling element (“low”), similarly to black-and-white photography. The light is scattered by the silver grains and the transmission is thereby attenuated.

A light-scattering bead represents another possibility for a signaling element.

Quantitative information can be obtained according to the invention by multiple determinations and/or by graded concentrations of the sensor element and/or analyte and subsequent statistics.

Reader

The invention furthermore relates to a reader which is optimized for the respective analysis support, and which, in an advantageous embodiment, allows semiautomatic or automatic positioning, passage and reading of the support. The support may also be automatically scanned.

An existing product from consumer electronics is preferably used as the reader, or new devices from common detection and mechanical units in combination. CD, DVD, magnetic-card or barcode readers may be mentioned here as examples.

In a preferred embodiment, the reader corresponds essentially to a manual magnetic-card reader in terms of its mechanics and handling. In this case, either the reader is installed mechanically fixed and the analysis support is passed through it, or the analysis support is placed fixed and the reader is moved linearly over the support by using corresponding mechanics.

In an advantageous embodiment of the invention, the reader is based on optical detection. The reader consists essentially of a “photoelectric barrier” with one or two sides. Corresponding to the signal type, a suitable reader head is used for the detection. The optical reader may, for example but not exclusively, have the embodiments described below.

A particularly preferred embodiment employs a commercially available CD reader head, which operates by reflection (FIG. 7A). Alternatively, a modified CD reader head is used which, in contrast to the reader unit used in CD players, can operate with transmitted light as well, or only with transmitted light (FIG. 7B). In this case, the silvering necessary in a conventional CD may be obviated, which leads to inexpensive production of the analysis support.

The analysis support may advantageously be oriented in both directions, i.e. with the front side, i.e. the side coated with sensor elements, toward or away from the reader unit, for example a CD reader head. The appropriate orientation depends on the biochemical detection protocol to be carried out, the respective signaling element, general constraints, for example coverage of the test areas or the base material of the analysis support, and the detection method. In the case of a reflection measurement, for example, it is advantageous for a silvered front side with a light-scattering signaling element to be oriented toward the optical reader unit, because a scattering element on a rear side would not be picked up in this case. For encapsulated liquid delivery by means of channel structures on the analysis support, in the case of reflective signaling elements, however, orientation of the front side away from the reader unit is advantageous. Otherwise, the light would need to pass through the channel structures for a reflection measurement, so that contamination and interference effects due to the solutions in the structures could occur.

Another preferred embodiment employs a conventional barcode reader, when the detection fields are arranged on the support as parallel strips in a pattern similar to a barcode (FIG. 8A). In a refinement of this embodiment, a multiplicity of parallel detection fields with identical substances may be fitted next to one another on such a test strip. In this case, the test results are read as a function of angle, for example orthogonally to the reading direction of the barcode reader (FIG. 8B). This gives rise to the possibility of multiple measurement in a detection being carried out, in order to obtain statistical information. Alternatively, a test strip subdivided in such a way may have detection fields to contain different, gradually arranged concentrations of sensor elements, so that quantitative information is possible. In this way, detections with micro and macro dimensions can be carried out in any desired combination on a support.

The parallel tracks of detection fields on an analysis support may also advantageously be read in parallel with multi-focus optics, for example 7 parallel light beams, as already used sometimes in modern CD-ROM devices. The individual photodiodes of a CD reader head, which are used for the tracking, may also be employed as a detector. An HF filter will be used as an isolator in this case, in order to separate the read signal from the tracking signal. In principle, the detection may also be carried out two-dimensionally, for example with a camera. This is particularly advantageous in the case of an extended arrangement of format elements, for example in the case of a 2D barcode.

Just as suitable as CD reader heads for detectors are their successors, as used in CD-ROM, CD-R and DVD readers, as well as magneto-optical and magnetic detectors, linear CCD arrays or photodiode arrays.

The invention is not restricted to optical data acquisition. In particular, magnetic detection methods may be used as analysis supports because of their widespread use in consumer electronics. Magnetic cards in banking as well as tape drives and hard disks in the computer industry may be mentioned here as examples. The associated reader systems can be used as reader devices after minor adaptations to the geometry of the respective analysis support.

Inside the reader, a reader head uses a senso-electrical transducer to generate analog signals, which can be processed directly by digital conversion and subsequent microprocessor logic for use in an evaluation system.

The analysis support is evaluated in four steps: a) measurement of the signal level, for example by means of a CD pick up; b) digitization of the analog signals, i.e. production of a digital signal sequence with the aid of a threshold criterion; c) interpretation of the digital signal stream with the aid of predefined format structures; d) comparison with the signal sequences allowed within the format structure.

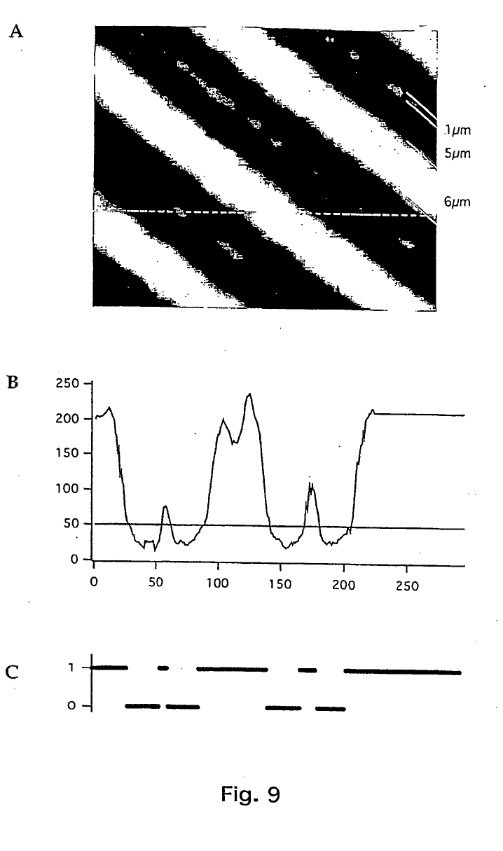

The digitization of the analog signal level is carried out in a standard device, for example in an electronic module with standard A/D converter logic. Image processing is obviated, because binary threshold criteria are provided by the device (FIG. 9). Synchronization of the data acquisition is carried out automatically with the aid of the predetermined sequence of detection, address and blank fields. In a more refined embodiment, special features of the signaling elements may be emphasized or suppressed with the aid of digital signal processing (DSP, microprocessor etc.), in order to obtain clear result information. This may be done, for example, by means of filtering or frequency analysis, by taking into account only signals with specific characteristics of the signaling elements and the format structures during the evaluation.

The incoming datastream is checked in an interpreter as to whether the chronological sequence of signal levels is permitted within the predetermined data format. Direct checking and error detection is thereby implemented. The signal sequence to be analyzed also contains the position information needed for the test. The received signal sequence is interpreted by comparison with the allowed sequences. A sequence which is allowed within the coding but is unexpected clearly indicates an error.

A series of individual tests on an analysis support can be carried out using a plurality of sequences of format elements arranged in rows. In the case of a CD, the format elements together with the detection fields are introduced directly into the track of pits and lands on the CD. The signal levels, the signal lengths, as well as the interpretation of the signal sequences, are given here by the Philips “Red Book” standard. The evaluation steps a) to c) are implemented in any standard CD player. The test is then evaluated directly by comparison of the predetermined input codeword with the output codeword which is read.

For evaluating the tests accommodated on the support, the subsequent computer system requires special software which is tailored to the respective analysis support. In an advantageous embodiment, this software may be accommodated in the data regions present on the same support. If the analysis support is correspondingly designed in terms of shape and function, it is possible to use readers such as floppy-disk or removable hard drives, CD-ROM players or comparable devices and their successors, with which the support is in principle compatible, in order to read the software. Advantageously, a plurality of readers may also be present in one device. The analysis software may also access databases optionally present on the support, in order to gain access to standard values which are required in the context of the specific analyses.

Applications

Owing to the ease of handling and the rapid and precise information about the detection results, the present invention can be used particularly advantageously in biochemical or biomedical detection methods. The invention furthermore relates to the substance combinations (“kits”) required for a detection. The following may be mentioned in detail:
hospital laboratories and genetic counseling for routine parallel diagnosis of genetic predispositions;
specialist medical practices for sensitive detection of pathogens, bacteria, viruses, autoimmune and tumor diseases, immunological overreactions and metabolic diseases;
pharmaceutical industry for mass screening to find pharmaceutically relevant active agents and quality control in active-agent production;
molecular biology in fundamental research for characterizing interactions in complex molecular mixtures;
plant genetics and hybrid development to cultivate commercially useful plants for agriculture, parallel analysis of plant features for example gene data;
environmental analysis for determining soil quality factors, contamination, occurrence and levels of microorganisms;
veterinary practice for simultaneously carrying out biochemical tests and unequivocal identification of the animal on site with the aid of an identification mark, for example a implantable microchip which can be read without contact through the skin, and which can be read with a simple transportable device;
point-of-care use for carrying out diagnostic methods on site, i.e. for a specialist practitioner or for outpatients, e.g. for routinely performed tests such as measuring blood sugar, blood lipid (LDL/HDL), determining immuno status etc.
food industry for detecting genetically modified organisms, monitoring microbiological or biochemical processes and quality control;
agriculture for developing agrochemicals.

Other uses of the analysis platform according to the invention are likewise possible, and are therefore included in the subject matter of the invention.

### EXEMPLARY EMBODIMENTS

### EXAMPLE 1

### Detection of C-reactive Protein by Means of Silver Precipitate Formation

A polycarbonate support is cleaned in water/ethanol (1:2) using ultrasound. A monoclonal antibody (Clone 5 (4C28), HyTest, Finland) against human C-reactive protein (CRP) is then printed onto defined regions of the support by means of a polydimethylsiloxane (PDMS) pad using a microcontact printing method. The pad and the support are in this case arranged with respect to one another, with the aid of an aligning unit, so as to ensure accurate positioning to within 5 μm. The support is then blocked for 30 min with a solution of 1% bovine serum albumin (BSA). After washing with buffer (PBS, 10 mM Na phosphate, 145 mM NaCl, 4 mM KCl, pH 7.4), the support is incubated for 30 min in a test sample containing the CRT protein. After rewashing with PBS, the substrate is incubated for 30 min with a second, biotinylated monoclonal antibody directed against a different epitope of CRP (Clone 7 (4C29), HyTest, Finland; in 1% BSA in PBS, 1:300 of the stock solution). In order to detect the binding which has taken place, by means of a sandwich immunoassay (FIG. 4), the support is incubated for 20 min with an antibiotin antibody conjugated to 1 nm colloidal gold particles, which is diluted by 1:400 in 1% BSA in PBS. A silver solution is added after washing. In an advantageous embodiment, this consists of 110 mg silver lactate, 850 mg hydroquinone (alternative: pyrogallol), 2.55 g citric acid monohydrate, 2.35 g trisodium citrate made up to 100 ml with water, which is freshly prepared immediately before the reaction. In order to ensure more uniform precipitate formation, the silver solution may be provided with further additives, for example UV blockers and reaction inhibitors such as gum arabic. After 2-4 min in the dark, the reaction is stopped by washing with distilled water and subsequently developed for 5 min with 3% (w/v) sodium thiosulfate in water. Alternatively, silver acetate may be used instead of silver lactate; in this case, the reaction does not take place in the dark, but for 15 min under normal daylight. In another advantageous embodiment, a commercial “silver enhancement” solution (e.g. from Sigma-Chemie, Munich) known to the person skilled in the art from immunohistology may be used instead. After rewashing with distilled water, the support is read in an essentially commercially available CD reader head.

### EXAMPLE 2

### Detection of C-reactive Protein by Means of Alkali Phosphatase

A support is prepared and processed in a similar way to Example 1. Instead of the antibiotin antibody, however, a streptavidin-alkali phosphatase conjugate is now added (Sigma-Chemie, Munich, 20 min, in 1% BSA in PBS, 0.5 mg/ml) and then washed. For signal development, the substrate is incubated for 10 min with ELF-97 phosphatase substrate (Molecular Probes, 5 mM in AP buffer: 150 mM NaCl, 1 mM MgCl_{2,} 1% BSA, 100 mm Tris-HCl, pH 9.5) and then thoroughly washed. The substrate converted by the enzyme precipitates at the site of the reaction. Alternatively, other substrates and other detection enzyme complexes may be used, for example alkali phosphatase with BCIP-NBT from Sigma-Chemie; streptavidin-peroxidase conjugate from Roche with 4-chloro-1-naphthol from Sigma-Chemie; peroxidase with DAP/Co from Sigma-Chemie.

### EXAMPLE 3

### Detection of Proteins via the Formation of Molecular Complexes

Biotinylated antibodies against a protein of interest are mixed with a patient's serum sample, which was previously diluted by 1:3 in PBS (see above). The mixture is centrifuged for 10 min at 13000×g, and the supernatant is applied to a detection support which, in a procedure similar to that in Example 1, is coated in particular places with an antibody that binds a different epitope on the protein to be detected from the serum. After thirty minutes of incubation at room temperature and washing three times in PBS, a freshly prepared mixture of core streptavidin and biotinylated ferritin in PBS cooled to 4° C. is applied to the detection support. The ferritin was in this case biotinylated with a kit according to the prior art, so that on average 4-10 biotin molecules are bound per ferritin tetramer. Within an incubation time of typically 30 minutes, when the level of the protein of interest has a certain value, large crosslinked complexes are formed at the places where the support is coated with the corresponding antibody (FIG. 5). These complexes can be detected optically, and their arrangement is subsequently read and detected in a similar way to that in Example 1.

### EXAMPLE 4

### Detection DNA Sequences via the Formation of Silver Precipitates

The DNA oligonucleotides, which have an amino group on the end, are immobilized on aminated polycarbonate supports (aminopropyltriethoxysilane, Fluka) by means of standard methods, for example Crosslinker BS3, Pierce. The single-stranded DNA is used as a sensor molecule for the binding of target-sequence DNA, which is biotinylated in the PCR reaction. After hybridization, the target DNA can be made visible by means of streptavidin-colloidal gold a and silver precipitation reaction, and detected with the aid of a CD reader head.

1. A method for detecting and/or quantifying at least one analyte in a sample on an analysis support, at least one defined sequence of fields being applied to the surface of the analysis support, characterized in that
a subset of the fields constitute detection fields;
a signal of one type is present on each field;
signals of at least one defined sequence of fields can be interpreted as a digital codeword;
sensor elements are applied to the detection fields in a controlled way;
analytes are brought in contact with the analysis support for the purpose of molecular interaction with the sensor elements on the detection fields;
signaling elements are localized on the detection fields when a molecular interaction has taken place;
one type of signal on the detection field where the molecular interaction has taken place is replaced by another type of signal in a predetermined way by the localization of signaling elements;
after the replacement of a signal of one type by a signal of another type on at least one detection field within a defined sequence of fields, the interpretation of this sequence of fields gives a different codeword than before the molecular interaction;
comparison of the codeword read after the detection with the known codeword before the detection gives the detection result.

2. The method as claimed in claim 1, characterized in that any replacement of a signal of one type by a signal of another type on a non-detection field within a defined sequence of fields is identified as an error during the interpretation.

3. The method as claimed in one of the preceding claims, characterized in that the signals on the fields can be read and interpreted in a format known from digital storage technology.

4. The method as claimed. in one of the preceding claims, characterized in that the signaling elements are designed and localized so that they can be read and interpreted in a format known from digital storage technology.

5. The method as claimed in one of the preceding claims, characterized in that a certain number of codewords have no detection fields and are used for separating and/or addressing sizable code blocks.

6. The method as claimed in one of the preceding claims, characterized in that the sensor elements of one type can be unequivocally assigned to at least one defined detection field on the analysis support, and vice versa.

7. The method as claimed in one of the preceding claims, characterized in that the fields on the analysis support a shaped as spots, strips, circles or spirals, or have another geometrical shape.

8. The method as claimed in one of the preceding claims, characterized in that individual fields on the analysis support are arranged in the form of a spot matrix or a circular, spiral, strip-shaped, linear or other geometrical or stochastic structure.

9. The method as claimed in one of the preceding claims, characterized in that sequences of defined fields which represent codewords are arranged successively in the form of a track on the analysis support.

10. The method as claimed in one of the preceding claims, characterized in that the tracks are arranged circularly, spirally, linearly or in another defined way on the analysis support.

11. The method as claimed in one of the preceding claims, characterized in that biologically active substances such as sugars, steroids, hormones, lipids, proteins, in particular monoclonal or polyclonal or recombinant antibodies, peptides, antigens of any type, haptens, DNA, RNA, as well as natural and artificial derivatives thereof, in particular aptamers and PNA, organic-chemical active agent libraries, cells, microorganisms, viruses or parts thereof, preparations and extracts from biological materials, metabolites and the like can be used as the sensor elements.

12. The method as claimed in one of the preceding claims, characterized in that any resonant processes such as absorption, fluorescence, phosphorescence, plasmon resonance, quenching etc., and nonresonant processes such as reflection, diffraction, scattering etc., from spectroscopy can be used to generate the signals.

13. The method as claimed in one of the preceding claims, characterized in that electromagnetic effects such as piezo, resonance shift, capacitance change, Hall effect, magnetic effects, electrical charge displacement etc. can be used to generate the signals.

14. The method as claimed in one of the preceding claims, characterized in that microspheres of any shape and size, such as metal, magneto, silica or fluorescence-labeled beads, fluorescent or radioactive labels as well as molecular complexes or aggregates, layers of precipitates, or dyestuffs can be used as signaling elements.

15. The method as claimed in one of the preceding claims, characterized in that biological objects such as cells, bacteria, pollens, virus particles or parts thereof can be used as signaling elements.

16. The method as claimed in one of the preceding claims, characterized in that bodies and coatings, in particular metal grains, are formed as signaling elements on an initiator, in particular an electron donor, coupled to analyte molecules, at the site of the interaction.

17. The method as claimed in one of the preceding claims, characterized in that a binding, polymerization, precipitation, deposition or color reaction, or other chemical or biological reactions, are used to form signaling elements.

18. The method as claimed in one of the preceding claims, characterized in that the signals generated by the signaling elements are digitized by means of a threshold criterion.

19. The method as claimed in one of the preceding claims, characterized in that the dimensions of the signaling elements can be adapted to the dimensions of the detection fields.

20. The method as claimed in one of the preceding claims, characterized in that the signaling elements are designed so that one and only one signaling element of is localized on each detection field.

21. The method as claimed in one of the preceding claims, characterized in that the fields and the signaling elements can have dimensions smaller than 10 μm, preferably smaller than 2 μm and in particular smaller than 1 μm.

22. The method as claimed in one of the preceding claims, characterized in that the analyte in the sample is quantified with the aid of calibration fields, defined threshold criteria and/or statistics via multiple determination.

23. The method as claimed in one of the preceding claims, characterized in that a synchronization track with standardized substances, surface coatings or other structures for calibrating the reader and the detection is applied to the analysis support.

24. The method as claimed in one of the preceding claims, characterized in that standard substances for positive or negative controls are applied to defined detection fields and/or to adjacently or successively arranged rows of such detection fields.

25. The method as claimed in one of the preceding claims, characterized in that different concentrations are sensor elements of one type are applied to defined detection fields and/or to adjacently or successively arranged rows of such detection fields.

26. The method as claimed in one of the preceding claims, characterized in that any desired combination of conventional data stores, such as magnetic strips, card chips, barcodes, CD-ROM or CD-R, are integrated on the analysis support.

27. The method as claimed in one of the preceding claims, characterized in that the software, databases, signatures of other information with any desired configuration is present on the analysis support.

28. The method as claimed in one of the preceding claims, characterized in that encoding or identification of the detection to be carried out on the analysis support is present on the analysis support, in the same format or in another separately applied format.

29. The method as claimed in one of the preceding claims, characterized in that the fields are designed and arranged so that they can be read by means of a commercially available barcode reader.

30. The method as claimed in one of the preceding claims, characterized in that the analysis support is comparable in its physical features, such a shape, material, optical density and material thickness, as well as handling, to a magnetic card known from mass storage technologies.

31. The method as claimed in one of the preceding claims, characterized in that the analysis support is comparable in its physical features, such a shape, material, optical density and material thickness, as well as handling, to a CD, CD-ROM or DVD known from mass storage technologies, or successors thereof.

32. The method as claimed in one of the preceding claims, characterized in that the fields are arranged in the form of a spirally applied CD data track on the analysis support.

33. The method as claimed in one of the preceding claims, characterized in that one or more writable data tracks are applied to the analysis support.

34. An analysis support, characterized in that it has at least one of the features described in the preceding claims.

35. A device for reading the analysis support described in claim 34, characterized by the following features:
instruments for transmitted-light and/or incident-light detection;
instruments for magnetic or electrical detection;
an instrument for automatically finding the detection fields;
an instrument for manually, semiautomatically and automatically feeding the analysis support through the device, together with suitable mechanical guidance;
an instrument for recording analog signals;
an instrument for digitizing analog signals;
an instrument for time- and position-resolved synchronization of the recording of analog and/or digital signals;
an instrument for error correction when reading and/or interpreting signals.

36. A kit, containing the essential substances for production of the analysis support described in claim 34.

37. A kit, containing the essential substances for carrying out one or more detections on an analysis support described in claim 34.
